(19) United States
(12) Patent Application Publication
(10) Pub. No.: US 2007/0224266 A1
(43) Pub. Date: Sep. 27, 2007

(54) Descarbonylethoxyloratadine containing pharmaceutical composition

(75) Inventors: Jack Aurora, Lorraine (CA); Brijesh Kumar Dave, Mississauga (CA)

Correspondence Address: BCF LLP;25th Floor, 1100 Rene-Levesque Boulevard West, Montreal, QC, H3B 5C9, US

(73) Assignee: PHARMASCIENCE INC.

(21) Appl. No.: 11/642,545

(22) Filed: Dec. 21, 2006

(30) Foreign Application Priority Data
Mar. 24, 2006 (CA) 2,541,045

(51) Int. Cl. A61K 31/473; A61K 9/20

(52) U.S. Cl. 424/464; 514/290

(57) ABSTRACT

A pharmaceutical composition wherein the pharmaceutically active ingredient (i.e. drug component) comprises Descarbonylethoxyloratadine and which further comprises an acidic element and a pharmaceutically acceptable stabilizer element.

The present invention relates to a pharmaceutical composition wherein the pharmaceutically active ingredient (i.e. drug component) comprises Descarbonylethoxyloratadine (or a pharmaceutically acceptable salt or polymoph form thereof). Descarbonylethoxyloratadine is also known as Desloratadine. Desloratadine is a derivative of loratadine and is chemically known as 8-chloro-6,11 dihydro-11-(4-piperidylidene)-5H-benzo[5,6]-cyclohepta[1,2-b]pyridine. It is to be understood herein that Desloratadine may be referred to herein simply as DCL. DCL is known as a long acting tricyclic histamine antagonist. Pharmaceutical compositions comprising DCL may be used to treat allergic reactions in mammals and in particular is indicated for the relief of nasal and non nasal symptoms of allergic rhinitis.

Pharmaceutical compositions comprising DCL may additionally include one or more known pharmaceutically acceptable substances that is/are not a medicinally active constituent(s) such as for example, carrier or diluent elements. However it is known that some types of such medicinally inactive constituent(s) may induce or facilitate decomposition of DCL leading to its discoloration. The color instability in the active DCL ingredient may, for example, be attributed to a very minute amount of degradation product, such as the N-formyl impurity of desloratadine. The N-formyl impurity of desloratadine may be formed due to the presence of a wide variety of medically inactive substances commonly used in tablet formulations. Such instability may for example be brought on by the presence in the pharmaceutical composition of an acidic medically inactive substance such as for example an acidic substance; an acidic substance may be an acidic excipient(s) which may have a pH of less than 7 in water, (e.g. a pH in range of 3 to 5). Acidic substances used in the formulation of pharmaceutical compositions include known substances such as for example, lactose, lactose monohydrate, sodium benzoate, microcrystalline cellulose, etc. Thus various formulations have been proposed for the purpose of providing a stable composition of Desloratadine which would purportedly avoid the incompatibility between Desloratadine and acidic substances; as mentioned such acidic substances may be acidic excipients such as lactose and other mono and disaccharides. Proposed stabilized compositions are for example described for example in U.S. Pat. No. 6,100,274, U.S. patent application Ser. No. 10/082,685 published under no. US 2002/0123504 as well as in PCT application no. PCT/IN2004/000399 published under no. WO 2005/065047.

It would be advantageous to be able to prepare alternate stabilized (e.g. tablet) formulation(s) comprising DCL such as for example a stabilized oral dosage form of Desloratadine. It would in particular be advantageous to have a DCL drug formulation which may inhibit the formation of DCL degradation materials and provide a safe and efficacious drug product for human consumption.

Thus the present invention provides a pharmaceutical composition for oral administration comprising a pharmaceutically active component and a pharmaceutically acceptable carrier component, wherein said pharmaceutically active component comprises a DCL element, wherein said pharmaceutically acceptable carrier component comprises an acidic element, and wherein said composition further comprises a DCL-protective amount of a pharmaceutically acceptable stabilizer element (i.e. member) selected from the group consisting of magnesium oxide, calcium oxide, aluminum oxide and mixtures thereof.

It is to be understood herein that the expression pharmaceutically active component characterize the composition as comprising a “therapeutically effective amount” of the DCL element. It is also to be understood herein that the expression “DCL element” includes DCL, a (suitable) pharmaceutically acceptable DCL salt, a (suitable) pharmaceutically acceptable polymorphic form of DCL and the like, including mixtures thereof. In accordance with the present invention the DCL element may for example comprise (or consist of) DCL.

In accordance with the present invention a pharmaceutical composition, as mentioned above, comprises a DCL-protective amount of a stabilizer element (i.e. member), i.e. any predetermined amount may be used which is able to provide the desired effect keeping in mind, for example, the amount of DCL present in the composition. However, in accordance with the present invention a pharmaceutical composition may, for example, comprise up to 10% (e.g. from 1% to 10%) by weight of said composition of said stabilizer element. In accordance with the present invention the stabilizer element may, for example, comprise from about 1% to about 5% (in particular from about 1% to about 2.65%; more particularly from about 1% to about 2.5%; e.g. from about 1% to about 2.25%;) by weight of the composition.

In accordance with the present invention the a pharmaceutically acceptable stabilizer element (i.e. member) may comprise (or even consist) of magnesium oxide.

In accordance with the present invention a pharmaceutical composition may comprise a pharmaceutically acceptable acidic excipient element. An acidic excipient element may, for example, be selected from the group consisting of lactose, microcrystalline cellulose (MCC), silicified MCC and mixtures thereof.

A pharmaceutical composition of the present invention may be formulated in any (known) suitable, desired or necessary manner. A pharmaceutical composition in oral dosage form (e.g. tablet form) may for example be formulated by using direct compression or wet granulation processes which are known in the art of making solid oral dosage forms.

A dosage form (e.g. tablet) of the invention may, for example, be produced in the following way: powders and/or granules may be mixed together using currently known production techniques. Thus as mentioned above a dosage form may be obtained by compression of a simple powder mixture comprising a matrix component and a pharmaceutically active component in dry powder form. A dosage form may also be obtained by compression of a mixture of components wherein the mixture comprises non-drug components in dry powder form and granules, the granules having been obtained from the dry granulation, wet granulation, compaction or extrusion of a simple mixture of a dosage form component(s) and a pharmaceutically active component in dry powder form (e.g. a dosage form body may be a tablet made-up of a single essentially uniform body (e.g. single layer)).

It is to be understood herein, that the reference to an “excipient” is a reference to a pharmaceutically acceptable ingredient or substance contained in a drug formulation that is not a medicinally active constituent and which may be a substance used as a diluent or vehicle for a drug.

It is to be understood herein, that if a “class”, “range”, “group of substances”, etc. is mentioned with respect to a particular characteristic (e.g. weight ratio, concentration, time, % by weight (e.g. % w/w), and the like) of the present invention, the present invention relates to and explicitly incorporates herein each and every specific member and combination of sub-classes, sub-ranges or sub-groups therein whatsoever. Thus, any specified class, range or group is to be understood as a shorthand way of referring to each and every member of a class, range or group individually as well as each and every possible sub-class, sub-range or sub-group encompassed therein; and similarly with respect to any sub-class, sub-ranges or sub-groups therein. Thus, for example, as mentioned herein

the mention that the composition comprises up to 10% by weight of the composition of a stabiliser element or member is to be understood herein as specifically incorporating each and every sub-range as well as each individual weight amount such as for example 1% to 10%, up to 5% 5% to 10%, 0.4%, 4.9%, 5%, 6%, 9% etc.;

and similarly with respect to any other parameters whatsoever.

It is in particular to be understood herein that for any group or range, no matter how defined, a reference thereto is a shorthand way of mentioning and including herein each and every individual member described thereby as well as each and every possible class or sub-group or sub-class of members whether such class or sub-class is defined as positively including particular members, as excluding particular members or a combination thereof; for example an exclusionary definition for a parameter or element may read as follows: “provided that when one of A and B is -Z and the other is W, -Z may not be R”.

DCL is for example described in U.S. Pat. No. 4,659,716 (the entire contents of which is incorporated herein by reference).

It is to be understood herein that the expression “pharmaceutically acceptable salt”, “pharmaceutically acceptable salts” and the like refers to a salt prepared from pharmaceutically acceptable non-toxic acid or base including inorganic acids or bases or organic acids or bases. Such DCL salts are for example described in U.S. Pat. Nos. 4,659,716 and 5,595,997 (the entire contents of these patents are incorporated herein by reference). Examples of inorganic acids are hydrochloric acid, hydrobromic acid, hydroiodic acid, sulfuric acid, and phosphoric acid. Organic acids may be selected, for example, from acids, such as formic acid, acetic acid, propionic acid, succinic acid, glycolic acid, glucuronic acid, maleic acid, furoic acid, glutamic acid, etc. U.S. patent application Ser. No. 432,387 published under no. 20050203116 provides a further description of salts of DCL (the entire contents of this patent document is also incorporated herein by reference).

Polymorphic forms of Desloratadine are for example described in U.S. Pat. No. 6,506,767. U.S. Pat. No. 6,962,924 also describes salts and polymorphs of Desloratadine hemifumarate. The entire contents of these patent documents is also incorporated herein by reference.

The phrase “therapeutically effective amount” is to be understood herein a referring to an amount of DCL element which provides a therapeutic benefit; for example, a therapeutic benefit in the treatment or management of allergic rhinitis.

The weight ratio, [namely X/(X+Y)], of the pharmaceutically active component (X) to the pharmaceutical composition (X+Y) as a whole (e.g. the whole tablet form) may be from 0.01 to less than 0.8; a weight ratio of 0.01 being for low drug loading medicaments and a weight ration of 0.8 being for high drug loading medicaments. For example the weight ratio of pharmaceutically active component (i.e. drug) to tablet in particular may be from 0.01 to 0.5 (e.g. the weight ratio of drug to tablet may be from 0.05 to 0.5 and more particularly the weight ratio of drug to tablet may be 0.06). As may be appreciated, in the above equation X/(X+Y), X is the pharmaceutically active component comprising a DCL element and Y is the rest of the components of the composition including the stabiliser element.

In accordance with the present invention a pharmaceutical acceptable carrier component may comprise one or more excipients and as desired or necessary one or more ancillary additives.

As used herein, in relation to excipients as well as other additives, the term “pharmaceutically acceptable” characterises the substances or compounds as compounds that are compatible with the other ingredients in a pharmaceutical formulation and are pharmaceutically acceptable to the subject when DCL is administered in therapeutically effective amounts.

An excipient (or diluent) may for example be a substance such as, for example, lactose, microcrystalline cellulose, sugars such as mannitol, sorbitol etc.

A pharmaceutical composition (e.g. a dosage form) of the present invention may for example optionally contain a pharmaceutically acceptable additive component comprising one or more ancillary additive members selected from the group comprising (e.g. consisting of)

a lubricant or anti-adherent (such as, for example, magnesium stearate sodium stearyl fumarate, zinc stearate, stearic acid, glyceryl behenate, glyceryl monostearate, etc.); a glidant (such as, for example, talc, colloidal silicon dioxide, or any other silica etc);

a binder (such as, for example, polyvinylpyrrolidone (PVP), starch, gelatin, ethyl cellulose, sodium carboxy methyl cellulose)

and

a disintegrant (such as, for example, Croscarmalose sodium, sodium starch glycolate, cross linked PVP, starch etc.).

As mentioned above pharmaceutically acceptable glidants, lubricants and other additives such as are well known to those of skill in the art (i.e. including, for example, those mentioned herein), may also be included in the composition, i.e. any such additive(s) may for example be included in the formulation in an amount of from 0.01% to 10% of the weight of the dosage form. For example a glidant is a substance that may be added during the granulation process in order for the granules to flow from a hopper onto a tablet press to the dies and for consistent and uniform fill.

In any event, it is to be kept in mind that any such ancillary additive(s), if present, is/are of course to be chosen and to be incorporated into, for example, a dosage form in amounts, keeping in mind the purpose thereof.

In the following examples, a commercially available pharmaceutically acceptable Desloratadine (DCL) was used. Similarly, in the following examples (commercially available) pharmaceutically acceptable materials were used for the stabilizer element and the carrier component, namely:

Magnesium: Magnesium oxide is selected as a stabilizer. It is an inorganic compound, which will provide basic pH (element) to the formulation.

Microcrystalline Cellulose PH 101: Microcrystalline cellulose (grade PH 101) was selected as a filler in formulations herein (i.e. Avicel PH 101—from FMC BioPolymer U.S.A.). It was selected over other Microcrystalline cellulose grades because of its good compressability.

Microcrystalline Cellulose PH 112: Microcrystalline cellulose (grade PH 112) was selected as a filler in formulations herein (i.e. Avicel PH 112—from FMC BioPolymer U.S.A.). It was selected over other Microcrystalline cellulose grades because of its low moisture content and desirable flow characteristics.
Starch 1500: Starch 1500 (a pregelatinized starch from Colorcon U.S.A.) was selected as a binder.

Starch 1500 LM (a pregelatinized starch from Colorcon U.S.A.): Starch 1500 LM (low moisture) was selected as a disintegrant. It was preferred over Starch 1500 due to low moisture content.

Hydroxypropyl Methylcellulose 2208 (i.e. Methocel K4M from Dow Chemicals U.S.A.): was selected for controlling drug release; K refers to HPMC (Hydroxypropyl Methylcellulose) polymer of 2208 substitution as type per USP (US Pharmacopia); 4M refers to 4000 mPₐₛ viscosity range as per USP.

Zinc Stearate: Zinc stearate was selected as lubricant.

Lactose anhydrous: Lactose anhydrous was selected as an additional filler.

### EXAMPLE 1

### Tablets Comprising Microcrystalline Cellulose

The composition of the tablets is as follows wherein the percentages are given as percentage of the overall weight of the composition or blend:

Formulation I composition:

| Ingredient | | |
|---|---|---|
| no. | Ingredient | % |
| 1 | Desloratadine (DCL) | 5.00 |
| 2 | Magnesium Oxide | 2.25 |
| 3 | Microcrystalline cellulose PH 112 | 81.75 |
| 4 | Starch 1500 LM | 10.0 |
| 5 | Zinc stearate | 1.0 |
| | Total | 100 |

As desired or necessary a tablet having a formulation composition in accordance with the present invention may further comprise a suitable (i.e. known) outer coating surrounding the compressed tablet core having a basic tablet formulation composition (e.g. a compressed tablet core wherein the basic tablet formulation composition is the Formulation I composition above may additionally include a coating). A compressed tablet core may for example, be coated with OPADRY Blue 03B90819 (Supplied by Colorcon) e.g. the coating may represent 2.5% w/w of the core tablet. The OPADRY Blue coating may, for example, be applied in known manner as for example as a dispersion in purified water.

Process of preparation of coated Formulation I composition: Direct Compression.

The process involved following steps:

| | |
|---|---|
| Step 1 | Mix Desloratadine and Magnesium oxide in Polyethylene bag. |
| Step 2 | Place Microcrystalline cellulose PH 112 in a V blender and add |
| | blend of step 1. in to it. Add Starch 1500 LM (Low moisture) |
| | in the V Blender. |
| Step 3 | Mix the ingredients of step 2 in V blender. |
| Step 4 | Lubricate the mixed blend of step 3 with Zinc stearate in |
| | V Blender. |
| Step 5 | Compress the final blend of step 4 on a rotary tablet press (i.e.. a |
| | Betapress from Manesty U.K.) with the target tablet weight of |
| | 85 mg and target tablet hardness of 9.0 kp. |
| Step 6 | Coat the compressed tablets with OPADRY Blue 03B90819 |
| | (Supplied by Colorcon) for 2.5% weight gain. |

A stability study provided a stability profile of the above mentioned coated Formulation I composition product which indicated that the product is a stabilized formulation. The stabilization study was conducted by packaging tablets of the above coated Formulation I composition in high density polyethylene (HDPE) bottles in the presence of a desiccant (trisorb 1 g) and of cotton batton and stored at 40° C./75% RH. Neither known nor unknown impurities are more than 0.1% (by weight) when stored at 40° C./75% RH for 1 month. After one (1) month the above coated Formulation I composition product (i.e. 20 tablets) were subjected to a High Performance Liquid Chromatographic assay to determine impurities; the solvent system comprised a Buffer pH 3.5 and acetonitrile in ratio of 75:25 v/v; column: USP L11 4.6×150 mm, 3 μm; and detector: 240 nm. No color change of the core tablets was observed at 1 month accelerated stability time point.

In Table 1 (as well as Tables 2, 3 and 4) below the compound impurities are as follows:

### Compound 1:

11-(4-piperidinylidene)-6,11-dihydro-5H-benzo(5,6)cyclohepta(1,2-b) pyridine

### Compound 2:

8-Bromo-11-(4-piperidinylidene)-6,11-dihydro-5H-benzo(5,6)cyclohepta(1,2-b)pyridine

### Compound 3:

8-Chloro-11-(4-piperidinylidene)-benzo(5,6)cyclohepta(1,2-b)pyridine

### Compound 4:

8-Chloro-11-(1-carboethoxy-4-piperidinylidene)-6,11-dihydro-5H-benzo(5,6)cyclohepta(1,2-b)pyridine

### Compound 5:

8-Chloro-6,11-dihydro-11-(N-methyl-4-piperidinylidene)-5H-benzo(5,6)cyclohepta(1,2-b)pyridine

### Compound 6:

8-Chloro-6,11-dihydro-11-(N-methyl-4-piperidinyl)-11-hydroxy-5H-benzo(5,6)cyclohepta(1,2-b)pyridine

### Compound 7:

8-Chloro-6,11-dihydro-5H-benzo(5,6)cyclohepta(1,2-b)pyridin-11-one

### Compound 8:

4,8-Dichloro-6,11-dihydro-5H-benzo(5,6)cyclohepta(1,2-b)pyridin-11-one

### Compound 9:

4,8-Dichloro-6,11-dihydro-11-(N-ethoxycarbonyl-4-piperidinylidene)-5H-benzo(5,6)cyclohepta(1,2-b)pyridine

### Compound 10:

8-Chloro-6,11-dihydro-11-(N-ethoxycarbonyl-4-piperidinyl)-11-hydroxy-5H-benzo(5,6)cyclohepta(1,2-b)pyridine

The impurity results for Formulation I are set forth in Table 1 below:

TABLE 1: Stability compilation of the above coated Formulation I composition product—The percentage of impurities is expressed as percentage by weight of the active ingredient Desloratidine; T=0 is the initial test date and 1 month refers to the analysis time after storage at 40° C./75% RH.

| | T = 0 | 1 Month |
|---|---|---|
| Impurities | | |
| Compound 1 | | * |
| Compound 5 | 0.002 | * |
| Compound 2 | | * |
| Compound 3 | 0.005 | * |
| Compound 6 | | * |
| Compound 7 | 0.005 | * |
| Compound 8 | 0.005 | * |
| Compound 4 | | * |
| Compound 9 | | * |
| Compound 10 | 0.02 | * |
| Unknown Impurity | | |
| RRT: 0.76 | | |
| RRT: 0.78 | | |
| RRT: 1.27 | | |
| RRT: 1.33 | | 0.03 |
| RRT: 1.70 | | |
| RRT: 2.57 | | |
| RRT: 2.61 | | |
| RRT: 2.69 | | 0.04 |
| RRT: 2.71 | | |
| RRT: 2.74 | | |
| RRT: 2.73 | | |
| RRT: 3.33 | | |
| Total unknown | 0.00 | 0.07 |
| Total related | 0.04 | 0.07 |

* Impurities below 0.03% was not mentioned in results

### EXAMPLE 2

### Tablets Comprising Microcrystalline Cellulose and Lactose Anydrous

The composition of the tablets is as follows wherein the percentages are given as percentage of the overall weight of the composition:
Formulation II composition:

| Ingredient no. | Ingredient | % |
|---|---|---|
| 1 | Desloratadine (DCL) | 5.88 |
| 2 | Magnesium Oxide | 2.65 |
| 3 | Microcrystalline cellulose | 79.41 |
| | PH 112 (Avicel PH 112) | |
| 4 | Lactose anhydrous | 5.29 |
| 5 | Starch 1500 LM | 5.59 |
| 6 | Zinc stearate | 1.18 |
| | Total | 100 |

The Formulation II composition was prepared in a manner analogous to that for the preparation of the Formulation I composition above.

A stabilization study provided a stability profile of the above Formulation II composition product which indicated that the product is a stabilized formulation. The stabilization study was also conducted by packaging tablets of the above Formulation II composition in high density polyethylene (HDPE) bottles in the presence of a desiccant (trisorb 1 g) and of cotton batton and stored at 40° C./75% RH. Neither known nor unknown impurities are more than 0.1% (by weight) when stored at 40° C./75% RH for 1 month. After one (1) month the above Formulation II composition product (i.e. 20 tablets) were subjected to a High Performance Liquid Chromatographic assay to determine impurities; the solvent system comprised a Buffer pH 3.5 and acetonitrile in ratio of 75:25 v/v; column: USP L11 4.6×150 mm, 3 μm; and detector: 240 nm. No color change of the core tablets was observed at 1 month accelerated stability time point.

The impurity results for Formulation II composition are set forth in Table 2 below:

TABLE 2: Stability compilation of Formulation II composition—The percentage of impurities is expressed as percentage by weight of the active ingredient Desloratidine; T=0 is the initial test date and 1 month refers to the analysis time after storage at 40° C./75% RH

| | T = 0 | 1 Month |
|---|---|---|
| Impurities | | |
| Compound 1 | 0.00 | 0.00 |
| Compound 5 | 0.00 | 0.00 |
| Compound 2 | 0.00 | 0.00 |
| Compound 3 | 0.00 | 0.00 |
| Compound 6 | 0.00 | 0.00 |
| Compound 7 | 0.00 | 0.00 |
| Compound 8 | 0.00 | 0.00 |
| Compound 4 | 0.00 | 0.00 |
| Compound 9 | 0.00 | 0.00 |
| Compound 10 | 0.00 | 0.00 |
| Unknown Impurity | | |
| RRT: 0.76 | 0.00 | 0.00 |
| RRT: 0.78 | 0.00 | 0.00 |
| RRT: 1.27 | 0.00 | 0.00 |
| RRT: 1.33 | 0.00 | 0.00 |
| RRT: 1.70 | 0.00 | 0.00 |
| RRT: 2.57 | 0.00 | 0.00 |
| RRT: 2.61 | 0.00 | 0.00 |
| RRT: 2.69 | 0.00 | 0.00 |
| RRT: 2.71 | 0.00 | 0.00 |
| RRT: 2.74 | 0.00 | 0.00 |
| RRT: 2.73 | 0.00 | 0.00 |
| RRT: 3.33 | 0.00 | 0.00 |
| Total unknown | 0.00 | 0.00 |
| Total related | 0.00 | 0.00 |

For the formulation compositions in examples 3 and 4 which follow, reference is made in the exemplified formulations III and IV to the additional presence of a OPADRY Blue coating in an amount of 2.5% w/w of the core tablet formulation. Reference(s) is (are) also made to the presence of a sufficient amount (q.s.) of water which is mentioned in relation to subsequently described the processes of making the respective tablet formulation compositions and not as an ingredient of the tablet formulation composition product.

### EXAMPLE 3

### Tablets Comprising Microcrystalline Cellulose and Lactose Anydrous

The composition of the tablets is as follows wherein the percentages are given as percentage of the overall weight of the composition:

(coated) Formulation III composition:

| Ingredient | | |
|---|---|---|
| no. | Ingredient | % |
| | Dry mix | |
| 1 | Desloratadine (DCL) | 5.88 |
| 2 | Magnesium Oxide | 2.65 |
| 3 | Microcrystalline cellulose PH 101 | 34.71 |
| | (Avicel PH 101) | |
| | Binder | |
| 4 | A Pregelatinized Starch (Starch 1500) | 2.94 |
| | Purified Water | q.s. |
| | Lubrication | |
| 5 | A Pregelatinized Starch (Starch 1500 | 2.65 |
| | LM) | |
| 6 | Lactose anhydrous | 5.29 |
| 7 | Microcrystalline cellulose PH 112 | 44.71 |
| | (Avicel PH 112) | |
| 8 | Zinc stearate | 1.18 |
| | Total (of core tablet) | 100 |
| 9 | Opadry Blue 03B90819 | 2.5% w/w of |
| | | core tablet |
| | Purified Water | q.s. |

The Formulation III composition was prepared by wet granulation process as described below:

Manufacturing process of formulation III composition:

| | |
|---|---|
| Step 1 | Portion (apprx. 13.33% by weight of total core tablet weight) of |
| | Microcrystalline cellulose PH 101 (of ingredient no. 3) was added in a high |
| | shear granulator and mixed for 1 min at slow impeller speed (e.g. 300 rpm |
| | impeller speed). |
| Step 2 | Desloratidine and magnesium oxide was introduced in a Polyethylene bag and |
| | dispersed (i.e. mixed/blended) for 3 min. This blend was introduced in high |
| | shear and mixed for 5 min at slow impeller speed(e.g. at 300 rpm impeller |
| | speed). |
| Step 3 | Remaining portion of Microcrystalline cellulose PH 101 (of ingredient no. 3) |
| | was passed through a 20 mesh (ASTM) screen then it was added in high shear |
| | granulator of step 2 and mixed for 5 min at slow impeller speed (e.g. 300 rpm |
| | impeller speed). |
| Step 4 | Pregelatinized starch (starch 1500) was dissolved in Purified water (15% starch |
| | 1500 in water). |
| Step5 | Step 3 blend was granulated in high shear granulator using granulating solution |
| | of step 4. |
| Step 6 | Wet granules were dried using fluid bed dryer at 55° C. inlet temperature till the |
| | LOD (Loss On Drying) (105° C., 2 g, 10 min) is achieved between 1.5–2.5%. |
| Step 7 | Dried granules were passed through co-mil equipped with 0.039″ screen and a |
| | conventional standard impeller. |
| Step 8 | Granules of step 7, Lactose Anhydrous, Microcrystalline cellulose PH 112 and |
| | Pregelatinized starch (Starch 1500 LM) were mixed in Polyethylene (PE) bag |
| | for 10 min. |
| Step 9 | Zinc stearate was mixed with blend of step 8 for 60 seconds in Polyethylene |
| | PE bag. |
| Step 10 | Final blend of step 11 was compressed using rotary tablet press (i.e.. a |
| | Betapress). |
| Step 11 | Opadry Blue 03B90819 was dispersed in Purified water. |
| Step 12 | Core tablets of step 10 were film coated up to 2.5% w/w weight gain using |
| | coating solution of step 11 in perforated coating pan. |

### EXAMPLE 4

### Tablets Comprising Microcrystalline Cellulose and Lactose Anhydrous and Prepared by Wet Granulation Process

The composition of the tablets is as follows wherein the percentages are given as percentage of the overall weight of the composition:

Formulation IV composition:

| Ingredient | | |
|---|---|---|
| no. | Ingredient | % |
| | Dry mix | |
| 1 | Desloratadine (DCL) | 5.88 |
| 2 | Microcrystalline cellulose PH 112 | 17.65 |
| 3 | Magnesium Oxide | 2.65 |
| 4 | Microcrystalline cellulose PH 112 | 17.06 |
| | Binder | |
| 5 | Pregelatinized starch (Starch 1500) | 2.94 |
| | Purified Water | q.s. |
| | Lubrication | |
| 6 | Pregelatinized starch (Starch 1500 LM) | 2.65 |
| 7 | Lactose anhydrous | 5.29 |
| 8 | Microcrystalline cellulose PH 112 | 42.70 |
| 9 | Hydroxypropyl Methylcellulose 2208 | 2.00 |
| 10 | Zinc stearate | 1.18 |
| | Total (of core tablet) | 100 |
| 11 | Opadry Blue 03B90819 | 2.5% w/w of |
| | | core tablet |
| | Purified Water | q.s. |

The composition of Formulation IV composition was prepared by wet granulation process as detailed below:

Manufacturing process of formulation IV composition:

| | |
|---|---|
| Step 1 | Microcrystalline cellulose PH 112 (ingredient 2) was passed through a 20 |
| | mesh (ASTM) screen then it was added in high shear granulator and mixed for |
| | 1 min at slow impeller speed. |
| Step 2 | Desloratidine and magnesium oxide was introduced in a Polyethylene bag and |
| | dispersed for 3 min. This blend was passed through a 20 mesh (ASTM) screen, |
| | introduced in high shear and mixed for 5 min at slow impeller speed. |
| Step 3 | Microcrystalline cellulose PH 112 (ingredient 4) was passed through a 20 |
| | mesh (ASTM) screen then it was added in high shear granulator of step 2 and |
| | mixed for 5 min at slow impeller speed. |
| Step 4 | Pregelatinized starch (starch 1500) was dissolved in Purified water. |
| Step5 | Step 3 blend was granulated in high shear granulator using granulating solution |
| | of step 4. |
| Step 6 | Wet granules were dried using fluid bed dryer at 42° C. inlet temperature till the |
| | LOD (105° C., 2 g, 10 min) is achieved between 1.5–2.5%. |
| Step 7 | Dried granules were passed through co-mil equipped with 0.039″ screen and |
| | standard impeller at slow speed (1200 rpm). |
| Step 8 | Lactose Anhydrous, Microcrystalline cellulose PH 112 (ingredient 8), |
| | Pregelatinized starch (starch 1500 LM) and Hydroxypropyl Methylcellulose |
| | 2208 were passed through a 20 mesh (ASTM) screen. |
| Step 9 | Granules of step 7 and powder of step 8 were mixed in Bin blender for 1200 |
| | seconds at 14 rpm. |
| Step 10 | Zinc stearate was passed through a 40 mesh (ASTM) screen and then it was |
| | dispersed in polyethylene bag with blend of step 9 for 30 seconds. |
| Step 11 | Dispersion of step 10 and blend of step 9 were mixed in Bin blender for 60 |
| | seconds then cover of bin blender were scrapped and again mixed for 60 |
| | seconds. |
| Step 12 | Final blend of step 11 was compressed using rotary tablet press. |
| Step 13 | Opadry Blue 03B90819 was dispersed in Purified water. |
| Step 14 | Core tablets of step 12 were film coated up to 2.5% w/w weight gain using |
| | coating solution of step 13 in perforated coating pan. |

A stabilization study provided a stability profile of the above Formulation III composition product which indicated that the product is a stabilized formulation. The stabilization study was also conducted by packaging tablets of the above Formulation III composition in high density polyethylene (HDPE) bottles in the presence of a desiccant (trisorb 1 g) and of cotton batton and stored at 40° C./75% RH. Neither known nor unknown impurities are more than 0.1% (by weight) when stored at 40° C./75% RH for 1 month. After one (1) month the above Formulation III composition product (i.e. 20 tablets) were subjected to a High Performance Liquid Chromatographic assay to determine impurities; the solvent system comprised a Buffer pH 3.5 and acetonitrile in ratio of 75:25 v/v; column: USP L11 4.6×150 mm, 3 μm; and detector: 240 nm. No color change of the core tablets was observed at 1 month accelerated stability time point.

The impurity results for the above Formulation III composition are set forth in Table 3 below:TABLE 3: Stability compilation of Formulation III composition—The percentage of impurities is expressed as percentage by weight of the active ingredient desloratidine; T=0 is the initial test date and 1 month refers to the analysis time after storage at 40° C./75% RH

| | T = 0 | 1 Month |
|---|---|---|
| Impurities | | |
| Compound 1 | 0.00 | 0.00 |
| Compound 5 | 0.00 | 0.00 |
| Compound 2 | 0.00 | 0.00 |
| Compound 3 | 0.00 | 0.00 |
| Compound 6 | 0.00 | 0.00 |
| Compound 7 | 0.00 | 0.00 |
| Compound 8 | 0.00 | 0.00 |
| Compound 4 | 0.00 | 0.00 |
| Compound 9 | 0.00 | 0.00 |
| Compound 10 | 0.00 | 0.00 |
| Unknown Impurity | | |
| RRT: 0.76 | 0.00 | 0.00 |
| RRT: 0.78 | 0.00 | 0.00 |
| RRT: 1.27 | 0.00 | 0.00 |
| RRT: 1.33 | 0.00 | 0.00 |
| RRT: 1.70 | 0.00 | 0.00 |
| RRT: 2.57 | 0.00 | 0.00 |
| RRT: 2.61 | 0.00 | 0.00 |
| RRT: 2.69 | 0.00 | 0.00 |
| RRT: 2.71 | 0.00 | 0.00 |
| RRT: 2.74 | 0.00 | 0.00 |
| RRT: 2.73 | 0.00 | 0.00 |
| RRT: 3.33 | 0.00 | 0.00 |
| Total unknown | 0.00 | 0.00 |
| Total related | 0.00 | 0.00 |

The stabilization study was also conducted by packaging tablets of the above Formulation IV composition in high density polyethylene (HDPE) bottles in the presence of a desiccant (trisorb 1 g) and of cotton batton and stored at 40° C./75% RH. Neither known nor unknown impurities are more than 0.1% (by weight) when stored at 40° C./75% RH for 1 and 2 months. After one and two months the Formulation IV composition product (i.e. 20 tablets) were subjected to a High Performance Liquid Chromatographic assay to determine impurities; the solvent system comprised a Buffer pH 3.5 and acetonitrile in ratio of 75:25 v/v; column: USP L11 4.6×150 mm, 3 μm; and detector: 240 nm. No color change of the core tablets was observed at 1 and 2 months accelerated stability time point.

The impurity results for Formulation IV composition are set forth in Table 3 below:TABLE 4:Stability compilation of Formulation IV composition—The percentage of impurities is expressed as percentage by weight of the active ingredient Desloratidine; T=0 is the initial test date and 1, 2 months refers to the analysis time after storage at 40° C./75% RH.

| | T = 0 | 1 Month | 2 Months |
|---|---|---|---|
| Assay (%) | 99.0 | 97.4 | 98.2 |
| Impurities | <0.05% | <0.05% | <0.05% |
| Compound 1 | <0.05% | <0.05% | <0.05% |
| Compound 5 | <0.05% | <0.05% | <0.05% |
| Compound 2 | <0.05% | <0.05% | <0.05% |
| Compound 3 | <0.05% | <0.05% | <0.05% |
| Compound 6 | <0.05% | <0.05% | <0.05% |
| Compound 7 | <0.05% | <0.05% | <0.05% |
| Compound 8 | <0.05% | <0.05% | <0.05% |
| Compound 4 | <0.05% | <0.05% | <0.05% |
| Compound 9 | <0.05% | <0.05% | <0.05% |
| Compound 10 | <0.05% | <0.05% | <0.05% |
| Unknown Impurity | | | |
| RRT: 2.26 | <0.05% | 0.05% | — |
| RRT: 3.93 | <0.05% | — | 0.06% |
| Total unknown | <0.05% | 0.05% | 0.06% |
| Total related | <0.05% | 0.05% | 0.06% |

### EXAMPLE 5

### pH Determinations for Formulations with Varying Amounts of MgO

A number of test were conducted to determine the amount of MgO which was needed to obtain a solution pH in the range of from about 5 to about 9.5. Thus, for the pH study, a series of powder blends was prepared using Desloratadine (5 mg), Microcrystalline cellulose PH 112 (74 mg), Starch 1500 LM, (20 mg), Zinc stearate(1 mg) and with varying amounts of Magnesium Oxide as set forth in table 2 below.

The pH investigation was carried out using by observing the pH change of 25 ml of 0.001 N Hydrochloric Acid, before and after addition into it of formulations of the present invention.

The method for pH determination was carried out as follows:
(1) 25 ml of 0.001 N Hydrochloric acid was placed in glass beaker and the pH determined using a pH meter.
(2) Thereafter one tablet or an equivalent amount of the composition was admixed to the acid in then glass beaker of step (1) and stirred with a glass rod until the composition was completely dispersed.
(3) the pH of the dispersion (obtained from step (2)) was then measured with the pH meter.

The results of the pH tests are set forth in the following table:

| | | pH of 0.001 N | pH of 0.001 N |
|---|---|---|---|
| Formulation | Amount of | Hydrochloric acid | Hydrochloric acid |
| composition | Magnesium oxide | before addition of | after addition of |
| No. | added | blend | blend |
| 1 | 0% w/w | 2.961 | 4.047 |
| 2 | 1% w/w | 3.041 | 5.193 |
| 3 | 2% w/w | 2.993 | 6.572 |
| 4 | 2.5% w/w | 2.996 | 9.091 |
| 5 | 5% w/w | 2.953 | 9.427 |

As may be seen from the above table MgO may advantageously be used in amounts of from about 1% to about 5% (in particular from about 1% to about 2.65%; more particularly from about 1% to about 2.5%; e.g. from about 1% to about 2.25%;) by weight of the composition.

1. A pharmaceutical composition for oral administration comprising a pharmaceutically active component and a pharmaceutically acceptable carrier component, wherein said pharmaceutically active component comprises a DCL element, wherein said pharmaceutically acceptable carrier component comprises an acidic element, and wherein said composition further comprises a DCL-protective amount of a pharmaceutically acceptable stabilizer element selected from the group consisting of magnesium oxide, calcium oxide, aluminum oxide and mixtures thereof.

2. A pharmaceutical composition as defined in claim 1 wherein said composition comprises up 10% by weight of said composition of said stabilizer element.

3. A pharmaceutical composition as defined in claim 1 wherein said stabilizer element comprises magnesium oxide.

4. A pharmaceutical composition as defined in claim 1 wherein said stabilizer element consists of magnesium oxide.

5. A pharmaceutical composition as defined in claim 2 wherein said stabilizer element comprises from 1% to 10% by weight of said composition and wherein said stabilizer element comprises magnesium oxide.

6. A pharmaceutical composition as defined in claim 2 wherein said stabilizer element comprises from 1% to 10% by weight of said composition and wherein said stabilizer element consists of magnesium oxide.

7. A pharmaceutical composition as defined in any one of claims 1 to 6 wherein said acidic element comprises an acidic excipient element.

8. A pharmaceutical composition as defined in claim 7 wherein said acidic excipient element is selected from the group consisting of lactose, microcrystalline cellulose and mixtures thereof.

9. A pharmaceutical composition as defined in any one of claims 1 to 8 wherein said composition is in the form of a tablet.

10. A pharmaceutical composition as defined in any of claims 1 to 9 wherein said stabilizer element comprises from 1% to 2.65% by weight of said composition.
